(19) United States
(12) Patent Application Publication
(10) Pub. No.: US 2013/0310725 A1
(43) Pub. Date: Nov. 21, 2013

(54) First Aid Pain Free BandAid

(71) Applicants: Richard L. Jerrells, Houston, TX (US); Sandy R. Jerrells, Houston, TX (US)

(72) Inventors: Richard L. Jerrells, Houston, TX (US); Sandy R. Jerrells, Houston, TX (US)

(21) Appl. No.: 13/870,651

(22) Filed: Apr. 25, 2013

Related U.S. Application Data
(60) Provisional application No. 61/687,463, filed on Apr. 25, 2012.

(51) Int. Cl. A61F 13/02

(52) U.S. Cl. CPC A61F 13/0246
USPC 602/43; 602/55

(57) ABSTRACT

The invention provides a uniquely designed band-aid that is pain free upon removal from skin surface by featuring gauze in between the top part of the strap and the bottom layer of the Punctured adhesive surrounded by a breakable tubing filled with alcohol or other liquid. The device provides a band-aid capable of being easily removed from the skin by saturating the adhesive prior to removal, thereby inflicting no pain on the user.

### CLAIM OF PRIORITY

This patent application claims priority under 35 USC 119 (e) (1) from U.S. Provisional Patent Application Ser. No. 61/687,463 filed Apr. 25, 2012, of common inventorship herewith entitled, “First Aid Pain Free BandAid.”

### FIELD OF THE INVENTION

The present invention pertains to the field of medical supplies, and more specifically to the field of medical band-aids.

### BACKGROUND OF THE INVENTION

The prior art has put forth several designs for medical bandages and components. Among these are:

U.S. Pat. No. 5,772,623 to Stephen Conte describes a flat, adhesive bandage having a lower, skin contacting surface coated with a first adhesive, and an upper surface partially coated with a second adhesive. At least one tab integrally extends from one edge of the bandage and is releasably adhered to the upper surface in complete covering relation to the second adhesive and in covering relation to an uncoated portion of the upper surface. When it is desired to remove the bandage from the skin, the tab may be grasped and lifted away from the upper surface. The tab may then be pulled in a direction to effectively remove the bandage. Therefore, it is not necessary to pry between the skin and the bandage's lower surface to effect removal of the bandage.

U.S. Pat. No. 6,855,743 to Nedeljko Vladimira Gvozdic describes reinforced, laminated, impregnated, and materials with composite properties as cross linked polyvinyl alcohol hydrogel structures in bulk or cellular matrix forms that can take essentially any physical shape, or can have essentially any size, degree of porosity and surface texture. They have a wide range of physical properties, unusual and unique combinations of physical properties and unique responses to stress fields, which allows for their use in many end use applications.

U.S. Pat. No. 7,396,976 to Marni Markell Hurwitz, Dave Narasimhan and Ernest D. Buff describes a bandage remains securely attached to the skin of a wearer during extended exposure to arid, humid or wet conditions. The bandage is readily removed from the attached condition upon application of pressure to its exterior surface. Adhesive portions of the contain pockets or microcapsules filled with an adhesive-inactivating ingredient. The pockets are formed in the backing layer. Microcapsules, if present, are incorporated in the adhesive. The adhesive inactivating ingredient comprises oil from vegetable source, mineral source or fatty acids. The wearer ruptures the pockets or microcapsules by applying pressure to the bandage above the adhesive portions. The adhesive-inactivating ingredient is thereby released at the skin-adhesive interface, permitting an easy, pain-free removal of the bandage.

None of these prior art references describe the present invention.

### SUMMARY OF THE INVENTION

It is an object of the present invention to provide a uniquely designed band-aid that is pain free upon removal from skin surface by featuring gauze in between the top part of the strap and the bottom layer of the punctured adhesive surrounded by a breakable tubing filled with alcohol. The design intent is to provide consumers with a band-aid capable of being easily removed from the skin by saturating the adhesive prior to removal, thereby inflicting no pain on the user.

### BRIEF DESCRIPTION OF THE DRAWINGS

FIG. 1 is a perspective diagonal top view showing a band-aid strap, gauze under top of the strap, tubing with alcohol and pull strings.

FIG. 2 is a perspective diagonal bottom view showing a band-aid strap with gauze under the strap, cotton padding for wound area, and pricks/holes in adhesive portion of strap to expose the gauze.

### DETAILED DESCRIPTION OF THE INVENTION

Minor nicks and cuts are not considered serious injuries, but these minor injuries do need to be treated with utmost concern and care. An untreated cut can result in infection. While it may be unlikely that an infection is left untreated, the entire situation is preventable from the moment of injury if the wound is properly cleaned and dressed. The band-aid is perhaps one of the most common first aid supplies available. The band-aid allows minor wounds to be treated and dressed with no professional medical assistance. Simple in form, the band-aid is a small bandage made of nylon fibers meshed with medical cloth; it features a small cotton padding and durable adhesive on its underside, allowing it to strongly hold to the skin. When a cut or scrape occurs, the wound is cleaned and a band-aid is placed over the wound to permit a sanitized healing process. The band-aid is great for treating minor wounds, but has a major downfall when the time comes to remove the band-aid. Removing a band-aid is a painful process as adhesive tightly pulls, the skin while the band-aid is being pulled off. The pain of removal may be inconsequential to a healthy adult, but it is very painful to a young child or an elderly hospital patient or to anyone else whose skin is delicate.

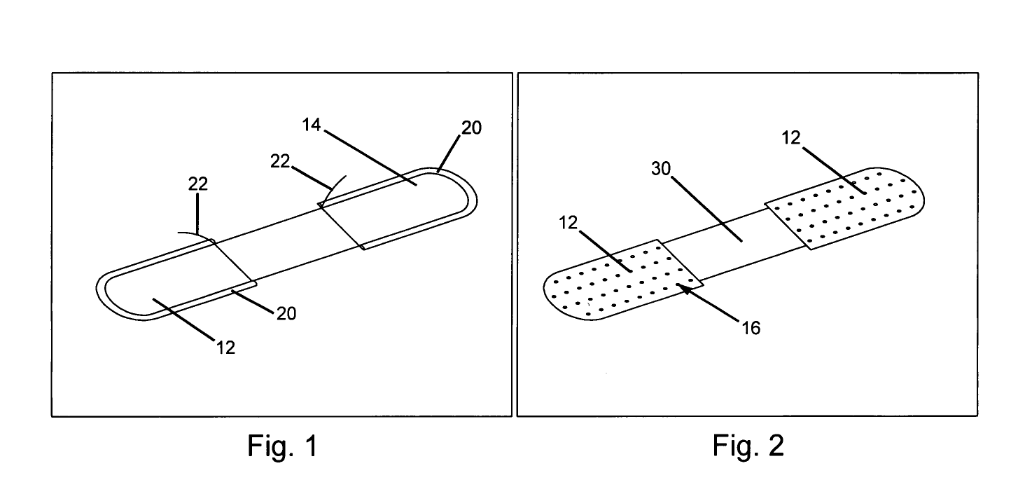

The present invention, hereinafter referred to as the First Aid Pain Free BandAid, is an improved variation of a band-aid that removes easily without damaging or hurting the skin. The First Aid Pain Free BandAid resembles a standard band-aid, in any suitable size, shape or material, featuring a lining of gauze 12 between the top layer of the band-aid strap 14, shown in FIG. 1, and the bottom layer 16 of the punctured adhesive. Bottom layer 16 is shown in FIG. 2. Surrounding the form or outer edge of the band-aid strap is a breakable tubing 20 containing a pocket of alcohol that, when released flows into the gauze where it saturates through the small punctures in the adhesive, diminishing the strength of the adhesive on the skin. The punctures in the bottom of the adhesive strap expose the gauze. The alcohol or liquid saturates the exposed gauze. The saturated gauze allows the band-aid to be painlessly removed from the skin. The release of the alcohol is performed in one of two ways. In the first embodiment, a user simply squeezes the tubing thereby releasing the alcohol into the gauze and through the punctured adhesive. In the second embodiment, a pull string 22, shown in FIG. 1, is incorporated into the design of the tubing where a user pulls the string, breaking the tubing which contains the alcohol. The pull string runs along the perimeter of the tubing. The pull string can comprise any suitable string such as cotton, nylon or other fiber, for example. The alcohol is then absorbed into the gauze where it saturates the punctured adhesive, loosening its grip from the skin. As an additional consideration in both embodiments, a liquid other than alcohol can be utilized in the tubing including but riot limited to witch hazel, astringents, baby oil, olive oil, coconut oil or other natural oils, concentrated or in solution, that loosen a strong adhesive from the skin while also protecting the skin at the same time.

FIG. 2 shows the bottom side of the device showing a central cotton pad 30 covers the wound and each side of the band-aid strap comprises the perforated adhesive 16, gauze layer 12 and liquid filled tubing 20. Liquid filled tubing 20 is shown in FIG. 1.

The First Aid Pain Free BandAid functions like a normal band-aid, carrying the same strength and reliability of those already on the market. More importantly, because the First Aid Pain Free BandAid features a gauze lining with alcohol filled tubing; users avoid any pain associated with the removal of the band-aid by simply releasing the alcohol prior to detachment. This is especially beneficial to young children or the elderly who have incredibly delicate skin as they no longer need to feel the sharp sting associated with a tightly bound band-aid from-their skin. The release of alcohol not only diminishes the pain of removing the First Aid Pain Free BandAid, but it also eliminates the chances of tearing or damaging the skin. Also, users no longer have to scrub away the sticky adhesive prior art band-aids leave behind, as the alcohol serves to remove most of it; a simple wipe, if necessary, cleans the remaining adhesive glue. The First Aid Pain Free BandAid is an enhanced band-aid designed to eliminate the pain associated with removing a band-aid from the skin.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A pain free band-aid device with releasably contained liquid to be released immediately before removing the band-aid, comprising a band-aid strap having a lining of gauze between a top layer of the band-aid strap and a bottom layer of punctured adhesive, further comprising a breakable tubing containing liquid, the liquid is releasable from the tubing and saturates the exposed gauze and flows through the punctures in the adhesive diminishing the strength of the adhesive on the skin and wherein the tubing runs along the outer edge of the band-aid strap.

2. The pain free band-aid device of claim 1 wherein the liquid is selected from the group consisting of alcohol, witch hazel, astringent, baby oil, olive oil, coconut oil and other natural oil, concentrated or in solution.

3. The pain free band-aid device of claim 1, wherein the liquid is released from the tubing by squeezing the tubing thereby releasing the liquid into the gauze and through the punctured adhesive.

4. The pain free band-aid device of claim 1, wherein the liquid is released from the tubing by pulling a release string which runs the perimeter of the tubing.

5. The pain free band-aid device of claim 1 further comprising a cotton pad section to cover a wound.

6. The pain free band-aid device of claim 5, wherein the liquid is released from the tubing by squeezing the tubing thereby releasing the liquid into the gauze and through the punctured adhesive.

7. The pain free band-aid device of claim 5, wherein the liquid is released from the tubing by pulling a release string which runs the perimeter of the tubing.

8. The pain free band-aid device of claim 4 wherein the string comprises cotton, nylon or other fiber.
